(19) United States
(12) Patent Application Publication
(10) Pub. No.: US 2007/0081961 A1
(43) Pub. Date: Apr. 12, 2007

(54) Extraction and use of collagen extracted from either a sea mammal or a bony fish

(76) Inventor: Adrien Beaudoin, Rock Forest (CA)

Correspondence Address: DLA PIPER US LLP;ATTN: PATENT GROUP, 1200 NINETEENTH STREET, NW, WASHINGTON, DC, 20036, US

(21) Appl. No.: 10/548,520

(22) PCT Filed: Mar. 10, 2004
(86) PCT No.: PCT/CA04/00377

§ 371 Date: Nov. 13, 2006

(30) Foreign Application Priority Data
Mar. 10, 2003 (CA) 2,422,906

(51) Int. Cl. A61K 38/17; C07K 14/00; A61K 8/64; C12N 5/06

(52) U.S. Cl. 424/70.14; 512/12; 530/356; 435/325

(57) ABSTRACT

A process for a cell culture wherein the cells are grown on a substrate, the improvement comprising the step of growing the cells on collagen substrate with the collagen having been extracted from an animal selected from the group consisting of a marine mamal and a bony fish. Preferred embodiments include those wherein the collage is either from salmon or a seal.

### FIELD OF THE INVENTION

The present invention relates to the extraction and use of collagen and more particularly, relates to the extraction and use of collagen extracted from either a sea mammal or a bony fish.

### BACKGROUND OF THE INVENTION

Collagen is the most abundant protein in mammals. Apart from water it is the nest major constituent of skin, cartilagineous tissues, blood vessels and teeth. It is generally found around the cells in tissues, forming with other proteins such as elastin and proteoglycans, the extracellular matrix. The composition of the latter varies from tissue to tissue. For example there is less elastin relative to collagen in the skin as compared to tendons where elastin is very important. The extracellular matrix exerts profound influences on many aspects of cell life, including morphology, differentiation and genetic expression, adhesion and proliferation. It is noteworthy that in the course of the aging process the extracellular matrix is considerably modified. It is well known that corporations in the cosmetic industry have taken advantage of the rejuvenating properties of collagen, in manufacturing and marketing of their products. Use of collagen can include in vitro cell culture, medical devices, cosmetic dermatological uses, etc.

In the past few years the use of collagens derived from bovine, porcine and aviary sources has become problematic especially with the appearance of prions in the case of mad cow disease (Creutfeld-Jacob Disease), and many types of animal viruses (including those closely related to SARS and potentially transmissible to humans). The need for alternative sources of collagen is pressing. Collagen is a family of fibrous proteins comprising more than 12 different Types. These Types are variations on a common theme with a basic structural similarity.

The most common types are:

Type I found in all the tissues, and more particularly the skin, tendons, bones, and cornea.

Type II found in cartilage, intervertebral discs, and vitreous bodies.

Type III found in the cardiovascular system and conjunctive tissues of the stroma, including fetal skin.

Type IV found in the basal membrane, the sub-adjacent basal membrane of endothelial cells and surrounding muscle tissues.

Type V found in the placenta, aminos, and chorion.

Different types of collagen, when used in cell culture, have somewhat different roles. Thus, Type I collagen tends to increase the cell attachment and the spreading on the flask surface, stimulates cells proliferation and allows the experimenter to use less serum in the medium. Type I also affects the adhesion and cell morphology and increases cell viability.

Type III collagens are known to increase cell attachment and modify the cell behavior.

Type IV favors cell attachment whereas the type V increases the proliferation of the cells and specifically endothelial cells.

For a more detailed analysis on the properties of the different types of collagen, one may refer to: Kuhn K (1987) The classical collagens: Type I, II, and III in Structure and Function of collagen Types (R. Mayne and R. E. Burgeson, eds) Academic Press NY p. 1. Collagen exists in three states:

i) Fibrous state, as in tendons;

ii) Cristalline state or tropocollagen;

iii) Denatured state as in gelatins.

Collagen molecules can be solubilized while keeping some intermolecular bounds. In 1955 Gallop P. M. (Arch.Biochem and Biophys.: 54.486-495) has isolated and partially characterized ichtyocol or fish collagen. He found that heating at 30° C. changed optical rotation −110° in two hours. He also noticed that molecular weight and viscosity were reduced. It was later found that the molecule was comprised of three helicoidal chains of about 100000 daltons forming the monomer and that the monomer was quickly denatured when temperature was raised explaining the reduction of viscosity. The absence of cross-linking between chains in the immature collagen allowed later the isolation and characterization of its basic structural unit, i.e. tropocollagen.

Tropocollagen has a molecular mass of 285 kDa. Type I collagen is a heterotrimer comprising two alpha-1 chains and another chain termed alpha-2. The other types of collagen have three identical chains, each one of them is comprised of about one thousand amino acids for a molecular mass of 285 kDa. The proportion of glycine residues is very high (one third). One finds also the highest proportion of proline encountered in any other known proteins. Finally two unusual amino acids are present, hydroxyproline and hydroxylysine. The glycine-proline-hydroxyproline sequence is repetitive.

Analysis of skin collagen Type 1 from the rainbow trout, a species belonging to the salmonoid family, revealed an unusual composition. Indeed collagens were found to be made of trimers of alpha-1, alpha-2, alpha-3, and 2 alpha-1 and 1 alpha-3 chains respectively. The occurrence of alpha-3 has been observed in bony fish only. The lower stability of these fish collagens seems to be related to the fact that there is a small number of Gly-Pro-Pro triplets and a large number Gly-Gly doublets thereby loosening the triple-helical structure.

(Ref: M. Saito, Y. Takenouchi, N. Kunisaki, S. Kimura)

Complete primary structure of rainbow trout type I collagen consisting of a1(1) a2(1) a3(1) heterotrimers European Journal of Biochemistry: 268(10) p 2817, May 2001

Some animal cells can be grown in suspension whereas others require an extracellular matrix to survive. Absence of such a layer for the latter type of adhering cells results in the loss of their morphological characteristics and eventually death.

On the market one finds two types of surface (flasks) on which cells adhere

i) Charged electrostatic polymer

ii) Collagen matrix

If one refers to the commonly available products, one finds that flasks covered with a collagen matrix are much more expensive than those covered with a polymer. In most cases it is more appropriate to use a collagen matrix than these polymers which are artificial to the cell. In this invention collagen from seal skin and collagen from salmon are proposed as a cell matrix for cell culture.

There are three different approaches commonly used to cultivate cells on a collagen matrix

1. On top of a collagen layer

2. Embedded in a collagen gel layer for three-dimensional culture. In this case, a cell-free bottom layer of collagen is usually placed in the culture flask to prevent attachment to the surface of the culture vessel. A top layer containing the cell is then placed on the bottom layer.

3. Between two collagen layers.

Cells which are routinely grown on collagen-coated surfaces, or collagen gels, include epithelial cells (e.g., hepatocytes, mammary epithelial cells, colonic epithelial cells, keratinocytes), some mesenchymal cells (e.g. endothelial cells) and neurons. The need for a collagen substrate is primordial to keep the integrity of stem cells.

The collagens of the present of the present invention are surprising in that they are ideally suited for cell culture as well as a number of other uses. Thus, it has typically been such in the industry, that, for cell culture, a membrane of gelatin must first be provided and which membrane is then coated with the collagen. The collagens extracted according to the present invention do not require such a membrane, but rather can be directly used on a flask or other substrate for the cell culture.

The collagens produced according to the present invention can also be used in medical devices. Thus, many such medical devices are implanted in the body and the use of the collagen of the present invention would assist in the growing of the cells as will be shown hereinbelow. Furthermore, the collagen derived from a seal does not carry any known risk of infection. Thus, seals are a furry salt water mammal and which have never been known to carry any infectious diseases.

In medical devices, the collagen would preferably be coated on the surface thereof. Indeed, it can even be used with medical sutures.

A further use of the collagens of the present invention would be for dermatological applications. As will be demonstrated hereinbelow, collagen has a great affinity for water and can act as an emulsifier.

In addition to the above, the collagens of the present invention could also be used, in admire, with other components which may be injected into the body. Thus, for example, it could readily be utilized in the case of broken bones.

Thus, as may be seen from the above, the collagen as derived from a marine mammal and in particular from a seal, shows excellent properties for cell culture. A salmon based collagen likewise shows interesting properties.

Protocol for cell culture
A. Flasks treated covered with collagene I (Biocoat collagen cell ware flasks)

No. Cat 354531, BD Biosciences (Rat tail tendon)

Falcon flasks without matrix

Flasks covered with salmon skin collagen

Flasks covered with seal skin collagen

Adhering cells (BJ=epithelial cells from human prepuce (ATCC)

Endothelial cells from human umbilical vein

Culture medium+fetal beef serum+trypsine.
B. Coating of culture flasks with collagen

2 ml of the experimental collagen was mixed with 2 ml of culture medium

A whitish precipitate formed which was evenly spread with a Q-tip on the flask bottom

Flasks were maintained for 20 minutes under the UV light in a laminar flow hood under UV light

About 2 million cells/10 ml were introduced in each flask incubation temperature was 37° C. Under (95% 02/5% CO2) atmosphere

### BRIEF DESCRIPTION OF THE DRAWINGS

In the drawings;

FIG. 1 shows an electro phosphorus of the collagens extracted from salmon skins and seal skins compared with other collagens.

FIG. 2 shows the cells of human skin without a matrix;

FIG. 3 illustrates cells of human skin cultivated without an extra cellular matrix;

FIG. 4 illustrates the growth of human skin cells utilizing seal collagen;

FIG. 5 shows the growth of human skin cells in a commercial collagen coated flask; and

FIG. 6 illustrates of growth of human skin cells on a matrix of collagen derived from salmon skin.

### DESCRIPTION OF THE PREFERRED EMBODIMENTS

TABLE 1
Influence of pH on solubility of fish and seal skin collagen

| Experiments | Seal | Fish |
|---|---|---|
| (number) | pH | pH |
| 1 | 4.85 | 4.33-5.21 |
| 2 | 4.69 | 4.30-5.31 |
| Mean | 4.77 | 4.32-5.26 |

Collagens are highly sensitive to pH variations. Seal collagen precipitates around pH 4.7, whereas salmon collagen precipitates between 4.32-5.26.

TABLE 2
Influence of ionic strength on collagen solubility of fish and seal skins.

| | Seal | Fish |
|---|---|---|
| Experiment | Ionic strength | Ionic strength |
| (number) | (M) | (M) |
| 1 | 0.150 | 0.647 |
| 2 | 0.155 | 0.647 |
| 3 | 0.150 | — |
| Mean | 0.152 | 0.647 |

Ionic strength is another factor that influences collagen solubility. Our results show a large variation in the NaCl concentration required to precipitate seal collagen as compared to salmon collagen. Results have been determined by two different methods, spectroscopy or by precipitate formation after centrifugastion. By spectroscopy, salmon collagen does not show a clear turn point, whereas seal collagen does. Salmon skin collagen requires a higher concentation of salt to precipitate, 0.647 M seal skin collagen precipitates at 0.152 M of NaCl

TABLE 3
Water content of seal collagen. (hydration)

| Experiments | Percentage |
|---|---|
| (number) | of water % |
| 1 (n = 3) | 97.3 |
| 2 (n = 2) | 98.1 |
| 3 (n = 2) | 97.9 |
| Mean | 97.8 |

Experiment 3. Was carried by lyophilisation instead of drying in section. The two techniques gave comparable results and shows that seal collagen absorbs approximately 98% water.

The procedure of collagen extraction from seal and fish skins was adapted from the method of Elsdale and Bard (Ref. Elsdale, T., Bard, J. (1972) Collagen substrata for studies on cell behaviour. J. Cel Biol. 54: 626-634) as described in the Second Edition of the “Culture of Animal Cells” by Ian R. Freshney, Edited by Alan R. Lis Inc., New York.

Collagen is soluble in liquid solution at low pH and salt concentration. Increasing the pH to 7.4 and increasing the ionic strength causes the collagen to gel.

The collagen was extracted as follows:

Materials
10× BME (Eagle Basal Medium)
7.5% solution of sodium bicarbonate
L-glutamine (200 mM)
Fetal calf serum
Nanopure water
0.142 M NaOH
0.5 M acetic acid

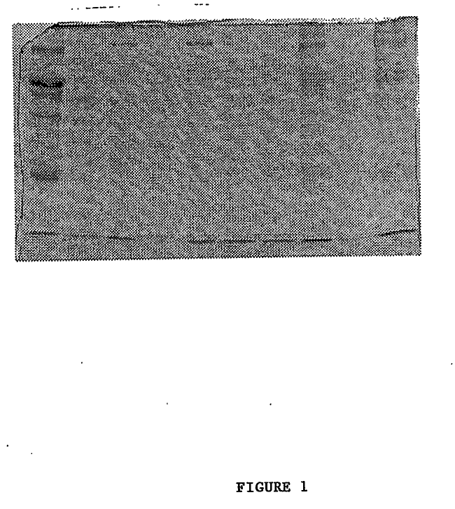

In FIG. 1 proteins were separated by sodium dodecylsufate polyacrylamide gel electrophoresis according to Laemmli (ref. Laemmli U K Cleavage of Structural Proeteins During the Assembly of the Head of Bacteriophage T4. Nature 227,680-685)

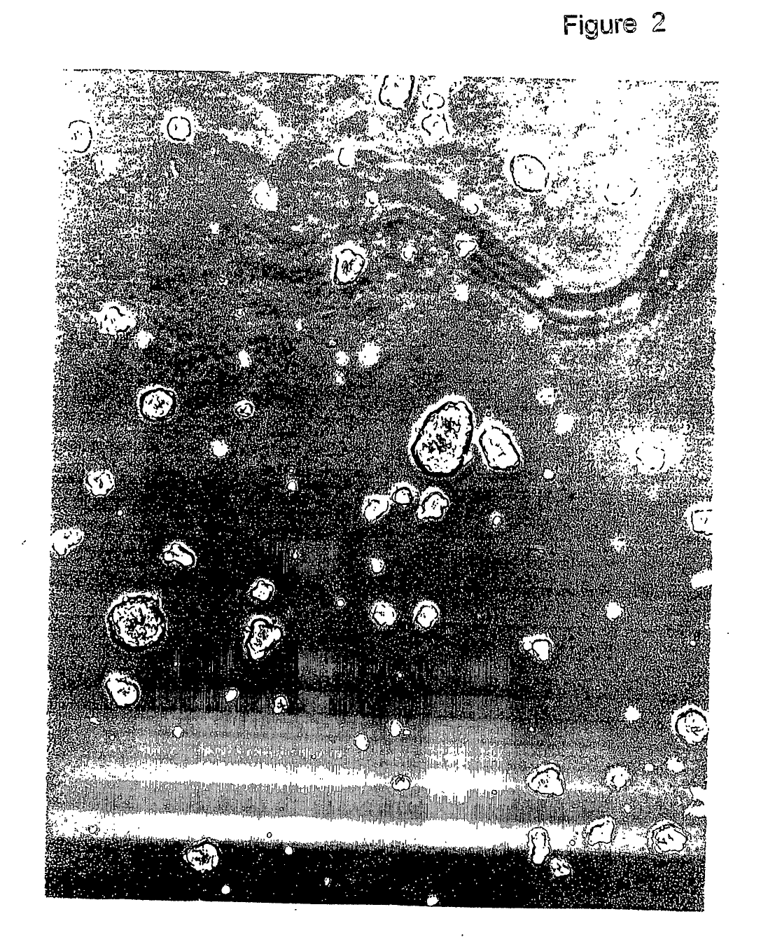

In FIG. 2, the cells were cultured in F12K medium supplemented with 10% fetal bovine serum (Sigma Chem. Co. St-Louis Mo. USA).

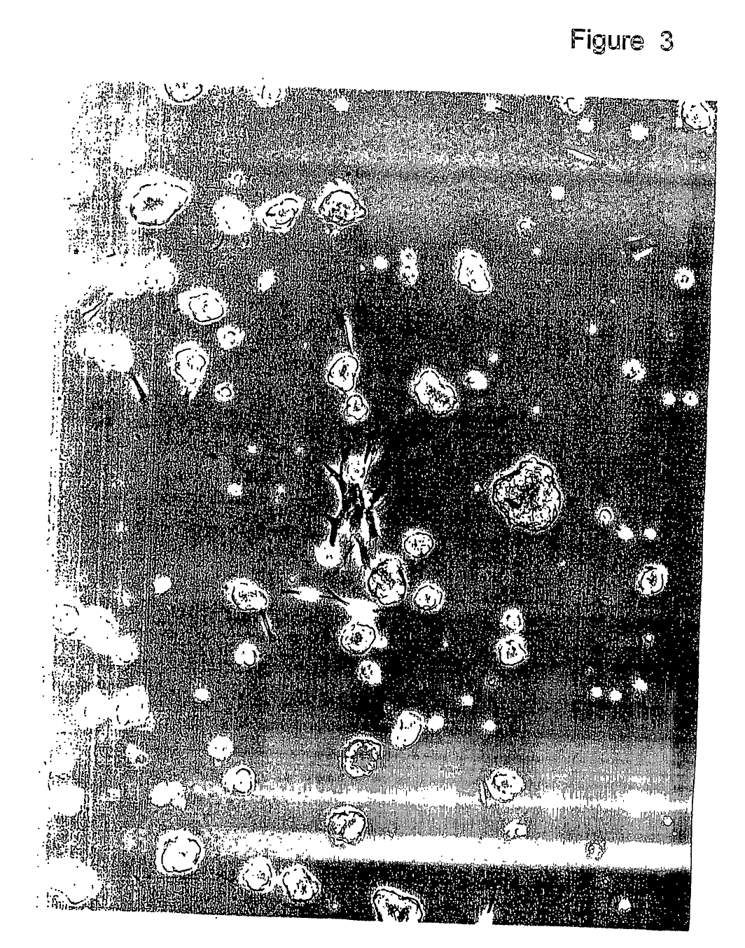

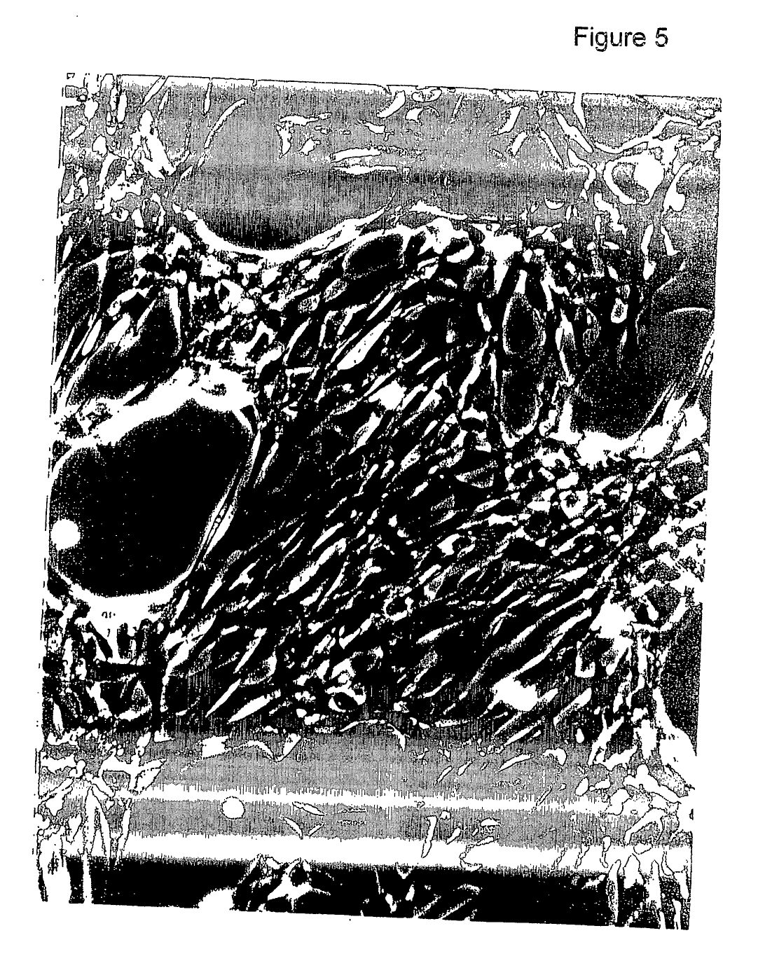

A similar culture medium was used for the experiments illustrated in FIGS. 3 to 6.

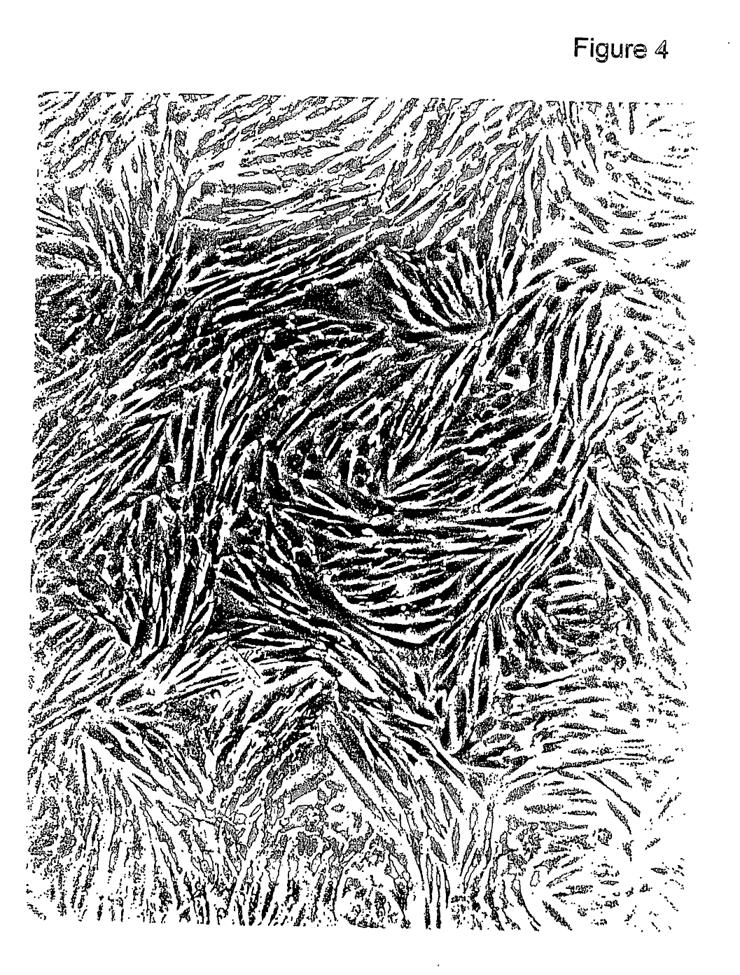

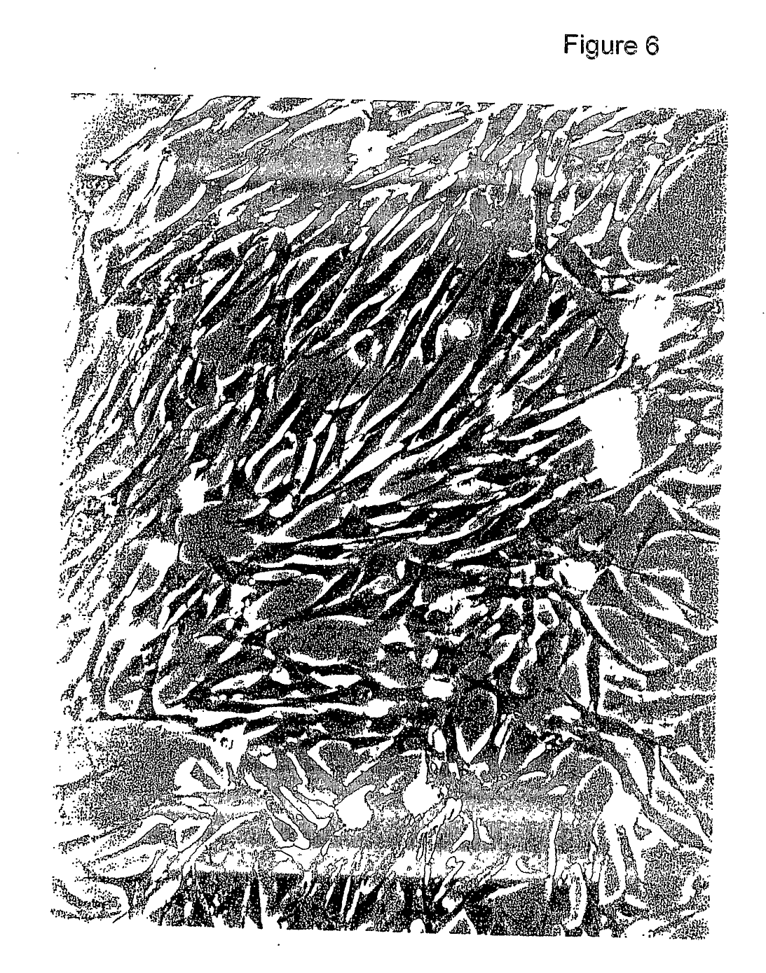

As may be seen from the figures, one can note that without a matrix of collagen, there is a lot of cellular debris and cells do not adhere to the flask. In FIGS. 4 and 6, which represent the cells grown using seal collagen and salmon collagen respectively, it will be noted the cells adhere well to the matrix and there is very little cellular debris.

On the other hand, with those cells grown in a flask with commercial collagen, it will be noted that the growth appears to be slower than the previous examples. Essentially similar results were obtained with human umbilical vein endothelial cells HUVEC ATCC CRL 1730. In the latter case heparin and endothelial cell growth supplement (from Sigma Chem Co.) Were added to the culture medium.

From the above, it will be seen that seal collagen can support the proliferation of epethetical cells like keratinocytes and endothelia cells in vitro and exhibits properties compatible with its use in medical devices. In particular, such medical devices can be mixed with the collagen.

I claim:

1. In a cell culture process wherein cells are grown on a substrate, the improvement comprising the step of growing said cells on a collagen substrate, said collagen being extracted from an animal selected from the group consisting of a marine mammal and a bony fish.

2. The improvement of claim 1 wherein said collagen is extracted from a marine mammal.

3. The improvement of claim 2 wherein said marine mammal is a seal.

4. The improvement of claim 3 wherein said collagen is a type 1 collagen.

5. The improvement of claim 1 wherein said collagen is extracted from a bony fish.

6. The improvement of claim 5 wherein said bony fish is salmon.

7. The improvement of claim 1 wherein said cells are cultivated mammalian cells.

8. The improvement of claim 7 wherein said cultivated mammalian cells are epithelial cells.

9. The improvement of claim 8 wherein said epithelial cells are keratinocytes.

10. In a container for the cultivation of cells, and wherein said container is at least partially coated with a collagen, the improvement wherein, said collagen is selected from a group consisting of collagens extracted from a sea mammal or a bony fish.

11. In a dermatological product, the improvement wherein said product contains collagen extracted from an animal selected from the group consisting of collagens extracted from sea mammals and a bony fish sea mammals and a bony fish.

12. Collagen when extracted from an animal selected from the group consisting of a marine mammal and a bony fish.
